SEQUENCE LISTING (40 sequences)

SEQ ID NO: 1 - protein, 39 aa, Artificial Sequence
  Exendin-4
HGEGTFTSDLSKQMEEEAVRLFIEWLKNGGPSSGAPPPS

SEQ ID NO: 2 - protein, 41 aa, Artificial Sequence
  H-(Lys)4-des Pro36, des Pro37 Exendin-4 (1-39)-NH2
KKKKHGEGTFTSDLSKQMEEEAVRLFIEWLKNGGPSSGAPS

SEQ ID NO: 3 - protein, 42 aa, Artificial Sequence
  H-(Lys)5-des Pro36, des Pro37 Exendin-4 (1-39)-NH2
KKKKKHGEGTFTSDLSKQMEEEAVRLFIEWLKNGGPSSGAPS

SEQ ID NO: 4 - protein, 38 aa, Artificial Sequence
  des Pro36 Exendin-4(1-39)
HGEGTFTSDLSKQMEEEAVRLFIEWLKNGGPSSGAPPS

SEQ ID NO: 5 - protein, 38 aa, Artificial Sequence
  des Pro36 [Asp28] Exendin-4(1-39)
HGEGTFTSDLSKQMEEEAVRLFIEWLKDGGPSSGAPPS

SEQ ID NO: 6 - protein, 39 aa, Artificial Sequence
  des Pro36 [IsoAsp28] Exendin-4(1-39)
  MISC_FEATURE (28)..(28): X=isoaspartate
HGEGTFTSDLSKQMEEEAVRLFIEWLKXGGPSSGAPPPS

SEQ ID NO: 7 - protein, 38 aa, Artificial Sequence
  des Pro36 [Met(O)14, Asp28] Exendin-4(1-39)
  MISC_FEATURE (14)..(14): X=isoaspartate
HGEGTFTSDLSKQXEEEAVRLFIEWLKDGGPSSGAPPS

SEQ ID NO: 8 - protein, 38 aa, Artificial Sequence
  des Pro36 [Met(O)14, IsoAsp28] Exendin-4(1-39)
  MISC_FEATURE (14)..(14): X=methionine oxide
  MISC_FEATURE (28)..(28): X=Isoaspartate
HGEGTFTSDLSKQXEEEAVRLFIEWLKXGGPSSGAPPS

SEQ ID NO: 9 - protein, 38 aa, Artificial Sequence
  des Pro36 [Trp(O2)25, Asp28] Exendin-4(1-39)
  MISC_FEATURE (25)..(25): X=tryptophan dioxide
HGEGTFTSDLSKQMEEEAVRLFIEXLKDGGPSSGAPPS

SEQ ID NO: 10 - protein, 38 aa, Artificial Sequence
  des Pro36 [Trp(O2)25, IsoAsp28] Exendin-4(1-39)
  MISC_FEATURE (25)..(25): X=tryptophan dioxide
  MISC_FEATURE (28)..(28): X=isoaspartate
HGEGTFTSDLSKQMEEEAVRLFIEXLKXGGPSSGAPPS

SEQ ID NO: 11 - protein, 38 aa, Artificial Sequence
  des Pro36 [Met(O)14 Trp(O2)25, Asp28] Exendin- 4(1-39)
  MISC_FEATURE (14)..(14): X=methionine oxide
  MISC_FEATURE (25)..(25): X=mtryptophan dioxide
HGEGTFTSDLSKQXEEEAVRLFIEXLKDGGPSSGAPPS

SEQ ID NO: 12 - protein, 38 aa, Artificial Sequence
  des Pro36 [Met(O)14 Trp(O2)25, IsoAsp28] Exendin-4(1-39))
  MISC_FEATURE (14)..(14): X=methionine oxide
  MISC_FEATURE (25)..(25): X=tryptophan dioxide
  MISC_FEATURE (28)..(28): X=isoapartate
HGEGTFTSDLSKQXEEEAVRLFIEXLKXGGPSSGAPPS

SEQ ID NO: 13 - protein, 44 aa, Artificial Sequence
  des Pro36 Exendin-4(1-39)-Lys6-NH2
HGEGTKTSDLSKQMEEEAVRLFIEWLKNGGPSSGAPPSKKKKKK

SEQ ID NO: 14 - protein, 50 aa, Artificial Sequence
  H-(Lys)6-des Pro36 [Asp28] Exendin-4(1-39)- Lys6-NH2
KKKKKKHGEGTFTSDLSKQMEEEAVRLFIEWLKDGGPSSGAPPSKKKKKK

SEQ ID NO: 15 - protein, 38 aa, Artificial Sequence
  des Asp28 Pro36, Pro37, Pro38Exendin- 4(1-39)-NH2
HGEGTFTSDLSKQMEEEAVRLFIEWLKGGPSSGAPPPS

SEQ ID NO: 16 - protein, 44 aa, Artificial Sequence
  H-(Lys)6-des Pro36, Pro38 [Asp28] Exendin-4 (1-39)-NH2
KKKKKKHGEGTFTSDLSKQMEEEAVRLFIEWLKDGGPSSGAPPS

SEQ ID NO: 17 - protein, 44 aa, Artificial Sequence
  H-Asn-(Glu)5des Pro36, Pro37, Pro38 [Asp28] Exendin-4(1-39)-NH2
NEEEEEHGEGTFTSDLSKQMEEEAVRLFIEWLKDGGPSSGAPPS

SEQ ID NO: 18 - protein, 44 aa, Artificial Sequence
  des Pro36, Pro37, Pro38 [Asp28] Exendin-4 (1-39)-(Lys)6-NH2,
HGEGTFTSDLSKQMEEEAVRLFIEWLKDGGPSSGAPPSKKKKKK

SEQ ID NO: 19 - protein, 50 aa, Artificial Sequence; identical to SEQ ID NO: 14
  H-(Lys)6-des Pro36, Pro37, Pro38 [Asp28] Exendin-4(1-39)-(Lys)6-NH2,

SEQ ID NO: 20 - protein, 50 aa, Artificial Sequence
  H-Asn-(Glu)5-des Pro36, Pro37, Pro38 [Asp28] Exendin-4(1-39)-(Lys)6-NH2,
NEEEEEHGEGTFTSDLSKQMEEEAVRLFIEWLKDGGPSSGAPPSKKKKKK

SEQ ID NO: 21 - protein, 50 aa, Artificial Sequence
  H-(Lys)6-des Pro36 [Trp(O2)25, Asp28] Exendin- 4(1-39)-Lys6-NH2
  MISC_FEATURE (31)..(31): X=tryptophan dioxide
KKKKKKHGEGTFTSDLSKQMEEEAVRLFIEXLKDGGPSSGAPPSKKKKKK

SEQ ID NO: 22 - protein, 38 aa, Artificial Sequence
  H-des Asp28 Pro36, Pro37, Pro38 [Trp(O2)25] Exendin-4(1-39)-NH2
  MISC_FEATURE (25)..(25): X=tryptophan dioxide
HGEGTFTSDLSKQMEEEAVRLFIEXLKGGPSSGAPPPS

SEQ ID NO: 23 - protein, 44 aa, Artificial Sequence
  H-(Lys)6-des Pro36, Pro37, Pro38 [Trp(O2)25, Asp28] Exendin-4(1-39)-NH2
  MISC_FEATURE (31)..(31): X=tryptophan dioxide
KKKKKKHGEGTFTSDLSKQMEEEAVRLFIEXLKDGGPSSGAPPS

SEQ ID NO: 24 - protein, 44 aa, Artificial Sequence
  H-Asn-(Glu)5-des Pro36, Pro37, Pro38 [Trp(O2)25, Asp28] Exendin-4(1-39)-NH2
  MISC_FEATURE (31)..(31): X=tryptophan dioxide
NEEEEEHGEGTFTSDLSKQMEEEAVRLFIEXLKDGGPSSGAPPS

SEQ ID NO: 25 - protein, 44 aa, Artificial Sequence
  des Pro36, Pro37, Pro38 [Trp(O2)25, Asp28] Exendin-4(1-39)-(Lys)6-NH2
  MISC_FEATURE (25)..(25): X=tryptophan dioxide
HGEGTFTSDLSKQMEEEAVRLFIEXLKDGGPSSGAPPSKKKKKK

SEQ ID NO: 26 - protein, 50 aa, Artificial Sequence; identical to SEQ ID NO: 21
  H-(Lys)6-des Pro36, Pro37, Pro38 [Trp(O2)25, Asp28] Exendin-4(1-39)-(Lys)6-NH2
  MISC_FEATURE (31)..(31): X=tryptophan dioxide

SEQ ID NO: 27 - protein, 50 aa, Artificial Sequence
  H-Asn-(Glu)5-des Pro36, Pro37, Pro38 [Trp(O2)25, Asp28] Exendin-4(1-39)-(Lys)6-NH2
  MISC_FEATURE (31)..(31): X=tryptophan dioxide
NEEEEEHGEGTFTSDLSKQMEEEAVRLFIEXLKDGGPSSGAPPSKKKKKK

SEQ ID NO: 28 - protein, 50 aa, Artificial Sequence
  H-(Lys)6-des Pro36 [Met(O)14, Asp28] Exendin-4 (1-39)-Lys6-NH2
  MISC_FEATURE (20)..(20): X=methionine oxide
KKKKKKHGEGTFTSDLSKQXEEEAVRLFIEWLKDGGPSSGAPPSKKKKKK

SEQ ID NO: 29 - protein, 38 aa, Artificial Sequence
  des Met(O)14 Asp28 Pro36, Pro37, Pro38 Exendin-4(1-39)-NH2
HGEGTFTSDLSKQEEEAVRLFIEWLKDGGPSSGAPPPS

SEQ ID NO: 30 - protein, 44 aa, Artificial Sequence
  H-(Lys)6-desPro36, Pro37, Pro38 [Met(O)14, Asp28] Exendin-4(1-39)-NH2
  MISC_FEATURE (20)..(20): X=methionine oxide
KKKKKKHGEGTFTSDLSKQXEEEAVRLFIEWLKDGGPSSGAPPS

SEQ ID NO: 31 - protein, 44 aa, Artificial Sequence
  H-Asn-(Glu)5-des Pro36, Pro37, Pro38 [Met(O)14, Asp28] Exendin-4(1-39)-NH2
  MISC_FEATURE (20)..(20): X=methionine oxide
NEEEEEHGEGTFTSDLSKQXEEEAVRLFIEWLKDGGPSSGAPPS

SEQ ID NO: 32 - protein, 44 aa, Artificial Sequence
  des Pro36, Pro37, Pro38 [Met(O)14, Asp28] Exendin-4(1-39)-(Lys)6-NH2
  MISC_FEATURE (14)..(14): X=methionine oxide
HGEGTFTSDLSKQXEEEAVRLFIEWLKDGGPSSGAPPSKKKKKK

SEQ ID NO: 33 - protein, 50 aa, Artificial Sequence; identical to SEQ ID NO: 28
  H-(Lys)6-des Pro36, Pro37, Pro38 [Met(O)14, Asp28] Exendin-4(1-39)-(Lys)6-NH2
  MISC_FEATURE (20)..(20): X=methionine oxide

SEQ ID NO: 34 - protein, 50 aa, Artificial Sequence
  H-Asn-(Glu)5 des Pro36, Pro37, Pro38 [Met(O)14, Asp28] Exendin-4(1-39)-(Lys)6-NH2
  MISC_FEATURE (20)..(20): X=methionine oxide
NEEEEEHGEGTFTSDLSKQXEEEAVRLFIEWLKDGGPSSGAPPSKKKKKK

SEQ ID NO: 35 - protein, 50 aa, Artificial Sequence
  H-Lys6-des Pro36 [Met(O)14, Trp(O2)25, Asp28] Exendin-4(1-39)-Lys6-NH2
  MISC_FEATURE (20)..(20): X=methionine oxide
  MISC_FEATURE (31)..(31): X=tryptophan oxide
KKKKKKHGEGTFTSDLSKQXEEEAVRLFIEXLKDGGPSSGAPPSKKKKKK

SEQ ID NO: 36 - protein, 38 aa, Artificial Sequence
  H-des Asp28 Pro36, Pro37, Pro38 [Met(O)14, Trp(O2)25] Exendin-4(1-39)-NH2
  MISC_FEATURE (14)..(14): X=methionine oxide
  MISC_FEATURE (25)..(25): X=tryptophan oxide
HGEGTFTSDLSKQXEEEAVRLFIEXLKGGPSSGAPPPS

SEQ ID NO: 37 - protein, 44 aa, Artificial Sequence
  H-Asn-(Glu)5-des Pro36, Pro37, Pro38 [Met(O)14, Trp(O2)25, Asp28] Exendin-4(1-39)-NH2
  MISC_FEATURE (20)..(20): X=methionine oxide
  MISC_FEATURE (31)..(31): X=tryptophan oxide
NEEEEEHGEGTFTSDLSKQXEEEAVRLFIEXLKDGGPSSGAPPS

SEQ ID NO: 38 - protein, 44 aa, Artificial Sequence
  des Pro36, Pro37, Pro38 [Met(O)14, Trp(O2)25, Asp28] Exendin-4(1-39)-(Lys)6-NH2
  MISC_FEATURE (14)..(14): X=methionine oxide
  MISC_FEATURE (25)..(25): X=tryptophan oxide
HGEGTFTSDLSKQXEEEAVRLFIEXLKDGGPSSGAPPSKKKKKK

SEQ ID NO: 39 - protein, 50 aa, Artificial Sequence; identical to SEQ ID NO: 35
  H-(Lys)6-des Pro36, Pro37, Pro38 [Met(O)14, Trp(O2)25, Asp28] Exendin-4(S1-39)-(Lys)6-NH2
  MISC_FEATURE (20)..(20): X=methionine oxide
  MISC_FEATURE (31)..(31): X=tryptophan oxide

SEQ ID NO: 40 - protein, 50 aa, Artificial Sequence
  H-Asn-(Glu)5-des Pro36, Pro37, Pro38 [Met(O)14, Trp(O2)25, Asp28] Exendin-4(1-39)-(Lys)6-NH2
  MISC_FEATURE (20)..(20): X=methionine oxide
  MISC_FEATURE (31)..(31): X=tryptophan oxide
NEEEEEHGEGTFTSDLSKQXEEEAVRLFIEXLKDGGPSSGAPPSKKKKKK

(19) United States
(12) Patent Application Publication
(10) Pub. No.: US 2019/0328969 A1
(43) Pub. Date: Oct. 31, 2019

(54) Drug Delivery Device

(71) Applicant: SANOFI, Paris (FR)

(72) Inventor: William Marsh, Warwick, Warwickshire (GB)

(21) Appl. No.: 15/776,155

(22) PCT Filed: Nov. 16, 2016
(86) PCT No.: PCT/EP2016/077796

§ 371 Date: May 15, 2018

(30) Foreign Application Priority Data
Nov. 16, 2015 (EP) 15306810.1

(51) Int. Cl. A61M 5/20; A61M 5/315

(52) U.S. Cl. CPC A61M 5/2033; A61M 2005/3126; A61M 5/31585

(57) ABSTRACT

A drug delivery device for dispensing a dose of a liquid medicament with a rotationally driveable expelling mechanism adapted to expel the dose of the liquid medicament from a medicament container, a round torsional drive spring adapted to apply a driving torque to the rotationally driveable expelling mechanism when energized by an external strain, wherein the round torsional drive spring encloses an interior space of varying shape and/or extension according to the level of external strain applied to the round torsional drive spring, and at least one restraining element defining a number of rest surfaces to internally support the round torsional drive spring increasingly with the level of external strain applied thereto, can be improved by providing the rest surfaces with an outer shape as to essentially maintain the centre of the interior space throughout the entire variation in external strain applicable to the round torsional drive spring.

### CROSS REFERENCE TO RELATED APPLICATIONS

The present application is the national stage entry of International Patent Application No. PCT/EP2016/077796, filed on Nov. 16, 2016, and claims priority to Application No. EP 15306810.1, filed in on Nov. 16, 2015, the disclosures of which are expressly incorporated herein in entirety by reference thereto.

### TECHNICAL FIELD

The disclosure relates to a drug delivery device.

### BACKGROUND

WO 2014/170177 A1 describes a torsion spring-based automatic injection device for expelling settable doses of a liquid drug. The spring mechanism of the injection device comprises a housing assembly and a dose setting assembly being rotatable relative to the housing assembly and a round torsional drive spring encompassed there between, such that the round torsional drive spring is strained when rotating the dose setting assembly relative to the housing assembly. The round torsional drive spring is helically coiled, having a longitudinal direction and a number of consecutive windings wherein a distal winding has a distal end and a proximal winding has a proximal end. Each of the windings has an outwardly pointing surface together forming an outside surface of the round torsional drive spring being parallel to the longitudinal direction. Either the housing assembly or the dose setting assembly or both comprises a polymeric spring receiving arrangement, which arrangement comprises a first surface substantially parallel with the longitudinal direction of the round torsional drive spring for abutting the distal end or the proximal end of the helical torsion spring and which arrangement further comprises a second surface substantially parallel with the longitudinal direction of the round torsional drive spring for supporting the outwardly pointing surface of the at least distal winding or the at least proximal winding.

### SUMMARY

Certain aspects of the present disclosure can be implemented to provide an improved drug delivery device. Certain aspects of the present disclosure can be implemented as a drug delivery device according to claim 1. Exemplary embodiments of the disclosure are defined in the dependent claims.

Accordingly, a general embodiment drug delivery device for dispensing a dose of a liquid medicament, the drug delivery device comprises a rotationally driveable expelling mechanism adapted to expel the dose of the liquid medicament from a medicament container, a round torsional drive spring adapted to apply a driving torque to the rotationally driveable expelling mechanism when energized by an external strain, wherein the round torsional drive spring encloses an interior space of varying shape and/or extension according to the level of external strain applied to the round torsional drive spring, and at least one restraining element defining a number of rest surfaces to internally support the round torsional drive spring increasingly with the level of external strain applied thereto, characterized in that the shape of the rest surfaces is such as to essentially maintain the centre of the interior space throughout the entire variation in external strain applicable to the round torsional drive spring.

In more specific embodiment of the drug delivery device, the rotationally driveable expelling mechanism includes a threaded piston rod and a drive sleeve, wherein the drive sleeve is configured, when rotationally driven, to cause the piston rod to rotate relatively to threaded member, the threaded member being provided in fixed axial position relative to the medicament container and in engagement with the thread of the piston rod.

In another more specific embodiment of the drug delivery device the round torsional drive spring extends in an axial direction and is eccentrically fixed with respect to a longitudinal axis of the drug delivery device, whereby one of the spring ends is arranged on the at least one restraining element and is fixed at a mounting point, and the at least one restraining element tapers in the axial direction from the mounting point.

The at least one restraining element provides a protection of a function of the drive sleeve during dose setting and dose dispensing. In particular, the at least one restraining element generates an abutting surface for the round torsional drive spring, e.g. distal spring coils, adjacent to fixed axial ends of the round torsional drive spring. This protects the drive sleeve from a direct contact with the round torsional drive springs, in particular with the spring coils, when the drive sleeve moves axially during dose setting, as dispense is triggered and rotationally during dose dispense. The drug delivery device is therefore improved regarding efficiency and reliability.

In another more specific embodiment, during biasing of the round torsional drive spring, the one or more restraining elements are adapted to partially space apart the round torsional drive spring from the drive sleeve.

In a further exemplary embodiment, the round torsional drive spring comprises a plurality of spring coils surrounding the drive sleeve.

According to another aspect of the disclosure, a diameter of the round torsional drive spring decreases during dose setting, whereby the round torsional drive spring is increasingly strained.

In an exemplary embodiment, the at least one restraining element is arranged between an axial end of the round torsional drive spring and an axial end of the drive sleeve, whereby an outer diameter of the at least one restraining element is radially spaced from an outer circumference of the drive sleeve. In particular, the outer diameter of the at least one restraining element varies, e.g. decreases in a proximal direction.

In an exemplary embodiment, the at least one restraining element comprises a number of axial projections that generate the abutting surface for the spring coils. The number of axial projections may protrude parallel to a torque axis of the round torsional drive spring which is parallel to a longitudinal axis of the drug delivery device. For example, the at least one restraining element comprises three to six axial projections distributed about a circumference of the at least one restraining element.

The number of axial projections depends on mechanism constraints. It may be possible to arrange a single axial projection as a continuous circular ring or a large number of narrower axial projections around the circumference or a single narrow axial projection.

In an exemplary embodiment, a first restraining element is coupled to or configured as a part of a housing of the drug delivery device, wherein the first restraining element fixes a distal spring end and comprises a number of first axial protrusions protruding proximally away from the fixed distal spring end.

Furthermore, a second restraining element is coupled to or configured as a part of a number sleeve, wherein the second restraining element fixes a proximal spring end and comprises a number of second axial protrusions protruding distally away from the fixed proximal spring end.

The spring ends may be fixed by an engagement with a fixing recess comprising a ramp, wherein the spring ends comprise hooks to fix the spring ends into the corresponding recess.

The round torsional drive spring may be biased due to a rotational movement of the number sleeve with respect to the housing. Because the round torsional drive spring is fixed to the housing and the number sleeve, due to the relative rotation of the number sleeve, the round torsional drive spring twists around a torque axis. At least a number of spring coils, in particular the spring coils adjacent to the fixed spring ends, abuts to the axial projections when the round torsional drive spring increasingly strained, thereby radially approaching towards the torque axis.

The round torsional drive spring may be released from strain during dispensing the dose of medicament, wherein the drive sleeve rotates together with the number sleeve with respect to the housing due to the release of the round torsional drive spring.

Further scope of applicability of the present disclosure will become apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE FIGURES

The present disclosure will become more fully understood from the detailed description given herein below and the accompanying drawings which are given by way of illustration only, and thus, are not limitative of the present disclosure, and wherein:

FIG. 1 is a schematic longitudinal section of an exemplary embodiment of a drug delivery device,

FIG. 2 is a schematic perspective view of a round torsional drive spring,

FIG. 3 is a schematic perspective view of a first restraining element of a housing of the drug delivery device.

FIG. 4 is a schematic longitudinal section of a second restraining element of a number sleeve of the drug delivery device

FIGS. 5 to 7 are schematic longitudinal sections of a part of the drug delivery device comprising the round torsional drive spring, a drive sleeve, the first restraining element and the number sleeve,

FIG. 8 is a schematic perspective view of an interface area of the number sleeve and the drive sleeve,

FIG. 9 is a schematic perspective view of an exemplary embodiment of the drive sleeve and

FIG. 10 is a schematic side view of the drive sleeve according to FIG. 9.

Corresponding parts are marked with the same reference symbols in all figures.

### DETAILED DESCRIPTION

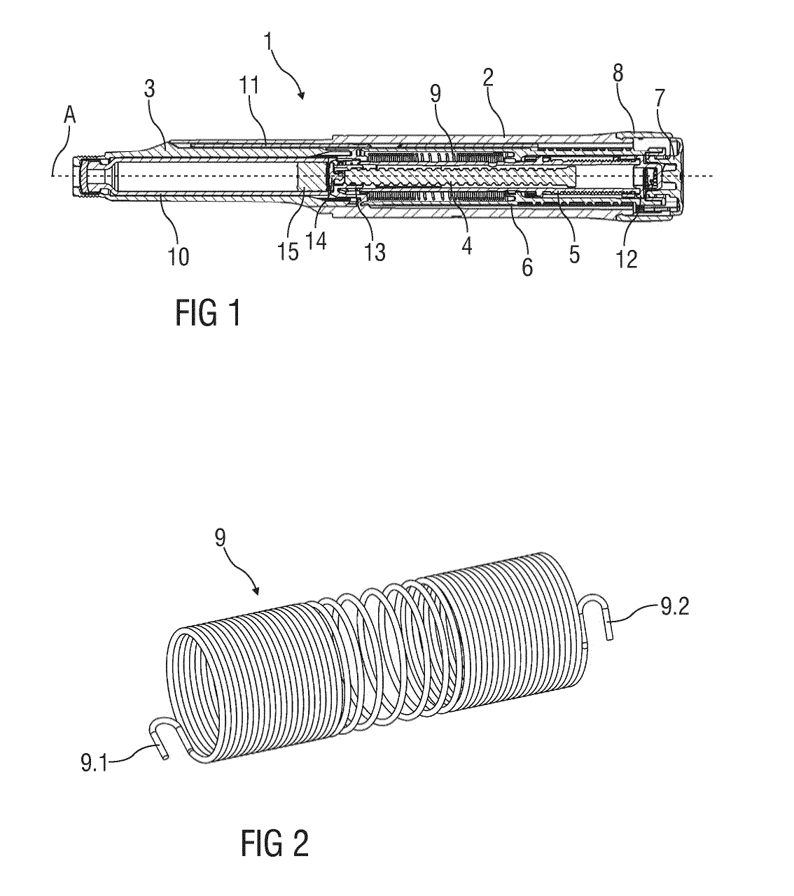

FIG. 1 shows a longitudinal section of an exemplary embodiment of a drug delivery device 1 in the form of a pen injection device.

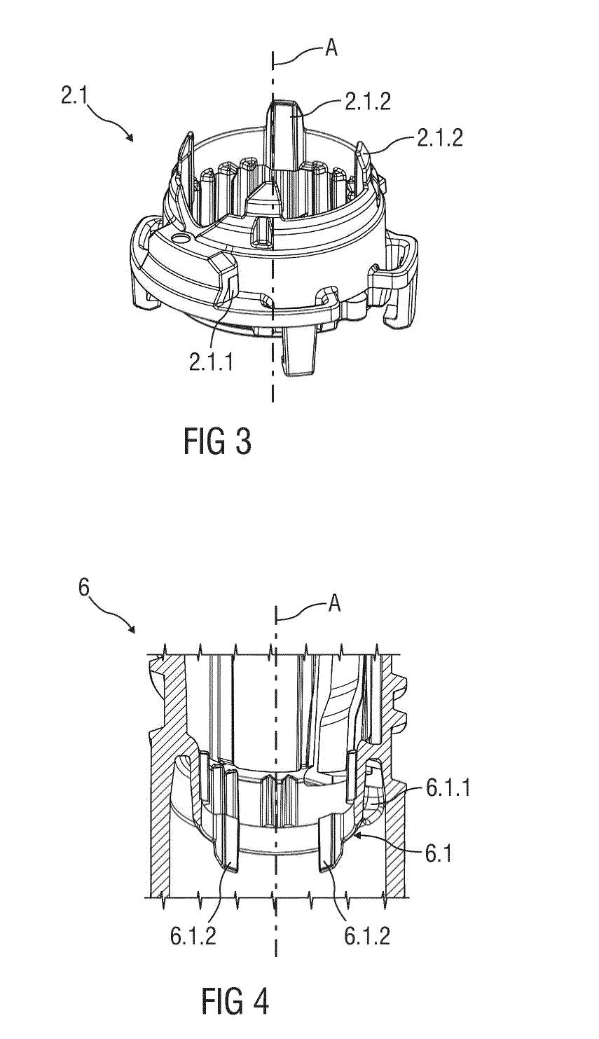

For protection of a driving function during dose setting and dose dispensing, the drug delivery device 1 comprises at least a drive sleeve 5 adapted to move a piston rod 4 in order to dispense the dose of medicament, a round torsional drive spring 9 adapted to move the drive sleeve 5 and at least one restraining element 2.1, 6.1, also shown in FIGS. 3, 4 in more detail, adapted to partially fix the round torsional drive spring 9 against axial and rotational movement with respect to the at least one restraining element 2.1, 6.1.

The drug delivery device 1 further comprises a housing 2, a cartridge holder 3, a number sleeve 6, a button 7, a dose selector 8, a cartridge 10, a gauge element 11, a clutch element 12, a clutch spring 13 and a bearing 14. A needle assembly (not shown) comprising a needle, a needle hub and a needle cover may be provided as additional components.

The drug delivery device 1 further comprises a longitudinal axis A extending from a proximal end P to a distal end D of the drug delivery device 1.

The housing 2 is configured as a substantially tubular body receiving the components of the drug delivery device 1 mentioned above. The cartridge holder 3 is arranged on a distal end of the housing 2 and attached thereto. The cartridge holder 3 receives the cartridge 10 from which a number of doses of a medicament may be dispensed by displacing a stopper 15 distally within the cartridge 10, wherein the stopper 15 is coupled to the piston rod 4. The distal end of the cartridge holder 3 may be provided with means for attaching a needle assembly (not shown), comprising a needle, a needle hub and a needle cover.

The piston rod 4 is threaded to the housing 2, wherein the piston rod 4 comprises an outer thread that engages a corresponding inner thread of the housing 2. A distal end of the piston rod 4 is engaged with the bearing 14 which acts on the stopper 15. The piston rod 4 is rotationally locked to the drive sleeve 5 such that the piston rod 4 moves axially with respect to the drive sleeve 5 when rotated.

The drive sleeve 5 has a substantially hollow cylindrical shape and encloses the piston rod 4. The drive sleeve 5 is proximally engaged to the clutch element 12 and distally engaged to the clutch spring 13. The drive sleeve 5 is further arranged within the number sleeve 6 and allowed to move distally with respect to the housing 2, the piston rod 4 and the number sleeve 6 against a bias of the clutch spring 13. The drive sleeve 5 is rotationally locked to the housing 2 during a dose setting and rotationally uncoupled from the housing 2 during dispensing a dose of medicament. Furthermore, the drive sleeve 5 is rotationally locked to the number sleeve 6 during dose dispensing.

The number sleeve 6 comprises a substantially tubular shape and is marked with a sequence of numbers on an outer circumference, which are visible through the gauge element 11. The number sleeve 6 is rotationally locked to the dose selector 8 during dose setting and thus rotated during dose setting via the dose selector 8. During dose dispensing, the number sleeve 6 is rotated together with the drive sleeve 5 by the round torsional drive spring 9, which is illustrated and explained in more detail in FIG. 2. The number sleeve 8 is further axially locked to the housing 2 and rotationally coupled to the button 7 during dose setting.

The button 7 forms the proximal end of the drug delivery device 1 and is permanently engaged to the dose selector 8. To activate a drug delivery mechanism, the button 7 is pressed distally as it is described further below.

The dose selector 8 is configured as a sleeve-like component with a ribbed outer surface in order to provide a grippable surface. The dose selector 8 is furthermore locked against axial movement with respect to the housing 2 and locked against rotational movement with respect to the button 7. A rotation of the dose selector 8 during dose setting charges the round torsional drive spring 9 in order to energise the drug delivery mechanism.

The round torsional drive spring 9 is inserted into the number sleeve 6, thereby enclosing a round interior space accommodating the distal part of the drive sleeve 5. The round torsional drive spring 9 comprises a distal spring end 9.1 being fixed to the housing 2 and a proximal spring end 9.2 being fixed to the number sleeve 6. The round torsional drive spring 9 is biased or charged during dose setting by rotating the dose selector 8 with respect to the housing 2. Because the dose selector 8 is rotationally locked to the number sleeve 6 and the number sleeve 6 is fixed to the proximal spring end 9.2 of the round torsional drive spring 9, the round torsional drive spring 9 is biased and decreases its diameter approaching a torque axis as described further below.

Further components of the drug delivery device 1 are the gauge element 11, the clutch element 12, the clutch spring 13 and the bearing 14.

The gauge element 11 comprises a generally plate- or band-like component having a central aperture (window) allowing viewing a portion of the number sleeve 6. The gauge element 11 is rotationally locked to the housing 2 but allowed to translate axially with respect to the housing 2.

The clutch element 12 is engaged to the number sleeve and rotationally locked thereto. The clutch element 12 is further locked against rotational movement to the button 7 at least during dose setting. The clutch element 12 provides an audible and/or tactile feedback for the user during dose setting and dose dispensing.

The clutch spring 13 may be a compression spring and defines the axial position of the drive sleeve 5, the clutch element 12 and the button 7, wherein the clutch spring 13 applies a force on the drive sleeve 5 in a proximal direction. This spring force is reacted via the drive sleeve 5, the clutch element 12 and the button 7, and further reacted by the dose selector 8 to the housing 2.

The bearing 14 is engaged to a distal end of the piston rod 4 and acts on the stopper 15 in a distal direction. The bearing 14 is axially locked and rotationally coupled to the piston rod 4. In order to perform a drug delivery process, the drug delivery device 1 may be operated according to the following exemplary method.

The user selects a variable dose of medicament by rotating the dose selector 8 clockwise, which generates an identical rotation of the number sleeve 6 with respect to the housing 2. A rotation of the number sleeve 6 causes the charging of the round torsional drive spring 9 as mentioned above, thereby increasing energy stored within. As the number sleeve 6 rotates, the gauge element 11 translates axially due to its threaded engagement, thereby showing the value of the dialled dose.

As a dose is set, the user may activate the drug delivery mechanism by depressing the button 7 in the distal direction, thereby initiating dose dispensation.

As a result, the button 7 and the dose selector 8 are rotationally disconnected from the number sleeve band the round torsional drive spring 9. The clutch element 12 and the drive sleeve 5 move axially together with the button 7, thereby engaging the drive sleeve 5 to the number sleeve 6 such that relative rotation between the drive sleeve 5 and number sleeve 6 is prevented. Furthermore, the engagement between the housing 2 and the drive sleeve 5 releases, thus the drive sleeve 5 is allowed to rotate and is driven by the round torsional drive spring 9 via the number sleeve 6 and the clutch element 12.

Rotation of the drive sleeve 5 causes rotation of the piston rod 4 which is axially translated due to its threaded engagement to the housing 2. Rotation of the number sleeve 6 causes the gauge element 11 to move axially back into a zero position, whereby a zero dose abutment (not shown) stops the drug delivery mechanism.

Since the bearing 14 is directionally engaged with the stopper 15, the bearing 14 does not rotate when the piston rod 4 rotates. Instead, the bearing 14 is axially translated during dose dispense.

If the user releases the button 7, the clutch spring 13 returns the drive sleeve 5 to an ‘at rest’ position (together with the clutch element 12 and the button 7), thereby engaging the drive sleeve 5 with the housing 2, preventing further rotation and stopping dose dispense. The user may then rotate the dose selector 8, so that the number sleeve 6 returns to the zero dose abutment.

FIG. 2 shows a perspective view of an exemplary embodiment of the round torsional drive spring 9.

The round torsional drive spring 9 is formed from a helical wire and has a number of coils, wherein a respective axial distance between adjacent coils in the centre of the round torsional drive spring 9 is increased with respect to an axial distance between adjacent coils of the remaining parts of the round torsional drive spring 9.

According to the illustrated embodiment, the distal spring end 9.1 and the proximal spring end 9.2 respectively comprise a hook for attachment to the housing 2 and the number sleeve 6 as described further below.

FIG. 3 shows a perspective view of an exemplary embodiment of a first restraining element 2.1 attached to the housing 2 or configured as a part of the housing 2.

The first restraining element 2.1 is a ring-like component and comprises a first fixing recess 2.1.1 for receiving the distal spring end 9.1.

The first restraining element 2.1 further comprises a number of first axial projections 2.1.2 projecting axially from an outer circumference of the first restraining element 2.1. The illustrated embodiment shows four first axial projections 2.1.2 distributed about the circumference. Alternatively, the first restraining element 2.1 may comprise less or more than the shown number of first axial projections 2.1.2.

The first axial projections 2.1.2 are arranged between the drive sleeve 5 and the round torsional drive spring 9 in the area of a terminal part of the drive sleeve 5, i. e. a distal part of the drive sleeve 5 as is illustrated in FIG. 5. The first axial projections 2.1.2 reduce a risk of damaging a function of the drive sleeve 5 due to an abutment of the coils of the round torsional drive spring 9 adjacent to the distal spring end 9.1 and the drive sleeve 5 during charging the round torsional drive spring 9. This may be the case without the first axial projections 2.1.2, since the geometry of the first fixing recess 2.1.1 locks the distal spring end 9.1 rotationally and radially, but not tangentially. During charging the round torsional drive spring 9, a diameter of the round torsional drive spring 9 decreases, whereby the coils adjacent the distal spring end 9.1 tend to pivot around an axis defined by the first fixing recess 2.1.1 until these coils abut the drive sleeve 5. A direct abutment between the round torsional drive spring 9 and the drive sleeve 5 would lead to an increase of a hysteresis of the round torsional drive spring 9 and resist axial movement and rotation of the drive sleeve 5. This may prevent reliable operation of the drive sleeve 5.

The first axial protrusions 2.1.2 extend in a direction parallel to a longitudinal extension of the round torsional drive spring 9, thereby parallel to a torque axis and thereby in the proximal direction away from the fixed distal spring end 9.1. The first axial protrusions 2.1.2 react radial forces on the drive sleeve 5 generated when the round torsional drive spring 9 is charged during dose setting. This protects the drive sleeve 5 from a direct contact with the round torsional drive spring 9, in particular when the drive sleeve 5 moves axially during dose setting and rotationally during dose dispense. Thus, the arrangement of the first axial protrusions 2.1.2 improves the drug delivery device 1 regarding an efficiency and reliability in holding a set dose.

The number of first axial projections 2.1.2 depends on mechanism constraints. Instead of a number of distributed first axial projections 2.1.2, it may be possible to arrange a single first axial projection 2.1.2 as a continuous circular ring or a large number of narrower axial projections around the circumference or a single narrow axial projection.

Furthermore, an axial length of the first axial protrusions 2.1.2 is essential to a correct function. If the axial length is too small, the coils of the round torsional drive spring 9 immediately adjacent to the distal spring end 9.1 may be deformed sufficiently to axially disengage the first axial protrusions 2.1.2 and thus directly abuts to the drive sleeve 5. If the axial length is too large, a bending strength of the first axial protrusions 2.1.2 is reduced and the coils of the round torsional drive spring 9 may deform the free ends of the first axial protrusions 2.1.2 radially inwards and thus contact the drive sleeve 5.

Additionally, the coils near the distal spring end 9.1 abut the first axial protrusions 2.1.2 during charging the round torsional drive spring 9, thus a number of active coils within the round torsional drive spring 9 reduces. Furthermore, angular deflection must be accommodated by a reduced number of active coils resulting in a higher mechanical stress in these remaining active coils. Thus, a minimum length of the first axial protrusions 2.1.2 is preferred in order to minimize a number of fixed coils. This conversely means: a number of remaining active coils is maximized and a peak of mechanical stress of the round torsional drive spring 9 during charging and release is minimized.

Furthermore, an outer surface of the first axial protrusions 2.1.2 is formed or shaped or structured to support the partial axial locking of the round torsional drive spring 9 to prevent the coils from slipping off them. In particular, the outer surface may be profiled, i.e. conical shape, stepped shape, friction surface.

Furthermore, an angular position of the first axial protrusions 2.1.2 relative to the first fixing recess 2.1.1 may affect proper function of the described features, particularly if a small number of first axial protrusions 2.1.2 is used. As described above, during charging the round torsional drive spring 9, the coils adjacent the distal spring end 9.1 tend to pivot around an axis defined by the first fixing recess 2.1.1 until these coils abuts to the drive sleeve 5. The angular position of the first axial protrusion 2.1.2 may be chosen knowing the point or points that the coils are most likely to abut the drive sleeve 5 relative to the fixing recess 2.1.1. For example the single first axial protrusion 2.1.2 may be best placed opposite the first fixing recess 2.1.1, where the effect of the diameter reduction will be greatest.

FIG. 4 shows a longitudinal section of a part of the number sleeve 6 comprising a second restraining element 6.1.

The second restraining element 6.1 may be a part of the sleeve 6 or a separate component attached thereto.

The second restraining element 6.1 comprises a second fixing recess 6.1.1 for receiving the proximal spring end 9.2 of the round torsional drive spring 9 similar to the first fixing recess 2.1.1.

The second restraining element 6.1 further comprises a number of second axial projections 6.1.2 projecting axially from an outer circumference of the second restraining element 6.1. The section view shows two of the four second axial projections 6.1.2, which are distributed about the circumference similar to the first axial projections 2.1.2. Alternatively, the second restraining element 6.1 may comprise less or more than four second axial projections 6.1.2.

The second axial protrusions 6.1.2 extend in a direction parallel to a longitudinal extension of the round torsional drive spring 9, thereby parallel to a torque axis and thereby in the distal direction away from the fixed proximal spring end 9.2. The second axial protrusions 6.1.2 react radial forces on the drive sleeve 5 generated when the round torsional drive spring 9 is charged during dose setting similar to the first axial projections 2.1.2.

The FIGS. 5 to 7 schematically show longitudinal sections of a part of the drug delivery device 1 comprising the distal end of the drive sleeve 5, the first restraining element 2.1, the distal spring end 9.1 of the round torsional drive spring 9 and a distal part of the number sleeve 6.

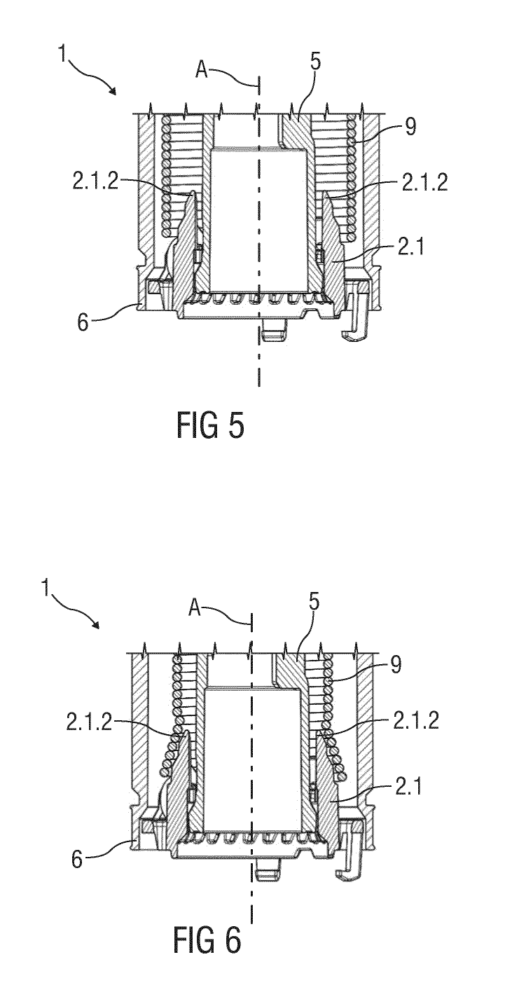

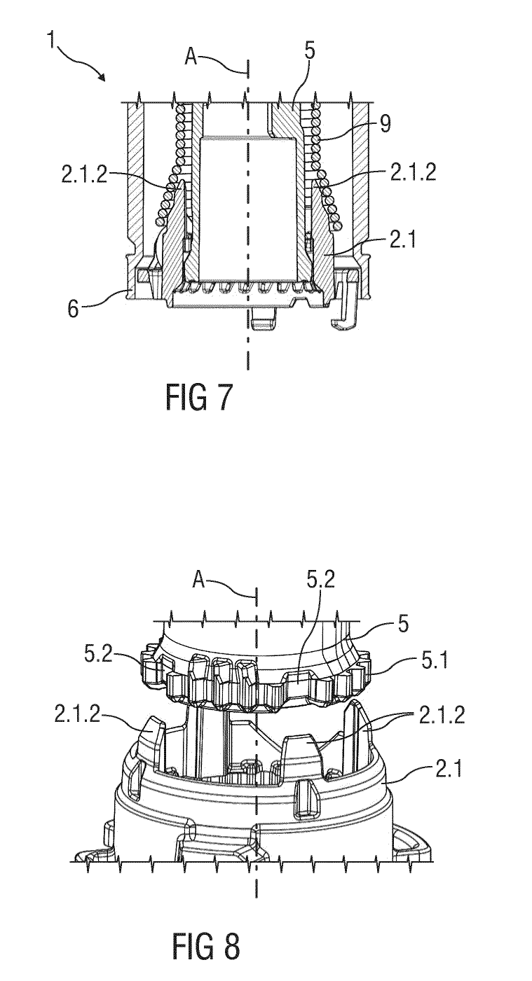

FIG. 5 illustrates the drug delivery device 1 during assembly, wherein the round torsional drive spring 9 is released. FIG. 6 illustrates the drug delivery device 1 with a selected minimum dose of medicament, wherein the round torsional drive spring 9 is charged by ten turns. FIG. 7 illustrates the drug delivery device 1 with a selected maximum dose of medicament, wherein the round torsional drive spring 9 is charged by fifteen turns. One turn corresponds with one turn of the dose selector 8 in the clockwise direction with respect to the housing 2.

The FIGS. 5 to 7 illustrate how the outer diameter of the first restraining element 2.1 in the area of the first axial protrusions 2.1.2 gradually reduces in order to guide the coils of the round torsional drive spring 9 towards the outer diameter of the drive sleeve 5, thereby maximising their bending strength. The same applies to the not shown second axial protrusions 6.1.2.

As a result, a longitudinal section profile of the round torsional drive spring 9 alternates from a conical shape to a cylindrical shape. This reduces a risk for the abutting coils to slide axially off the first and second axial protrusions 2.1.2, 6.1.2.

In order to improve the bending strength of the first axial protrusions 2.1.2, an inner diameter of the first restraining element 2.1 in the area of first axial protrusions 2.1.2 is reduced with respect to the inner diameter of a number of splined teeth which are distributed about an inner circumference of the housing 2 in order to engage the drive sleeve 5.

FIG. 8 shows a perspective view of an interface of the drive sleeve 5 and the first restraining element 2.1.

The drive sleeve 5 comprises a ring of splined teeth 5.1 that are distributed around an outer circumference of the distal end of the drive sleeve 5, thereby projecting radially outwards from the outer circumference. The distal end of the drive sleeve 5 further comprises a number of assembly recesses 5.2 that interrupt the ring of splined teeth 5.1 respectively. The number of assembly recesses 5.2 and their location respectively correspond with a number of first axial protrusions 2.1.2 and their location on the first restraining element 2.1. Thus, the first axial protrusions 2.1.2 engage the assembly recesses 5.2 during assembly. This allows an assembly of the drive sleeve 5 past the first axial protrusions 2.1.2.

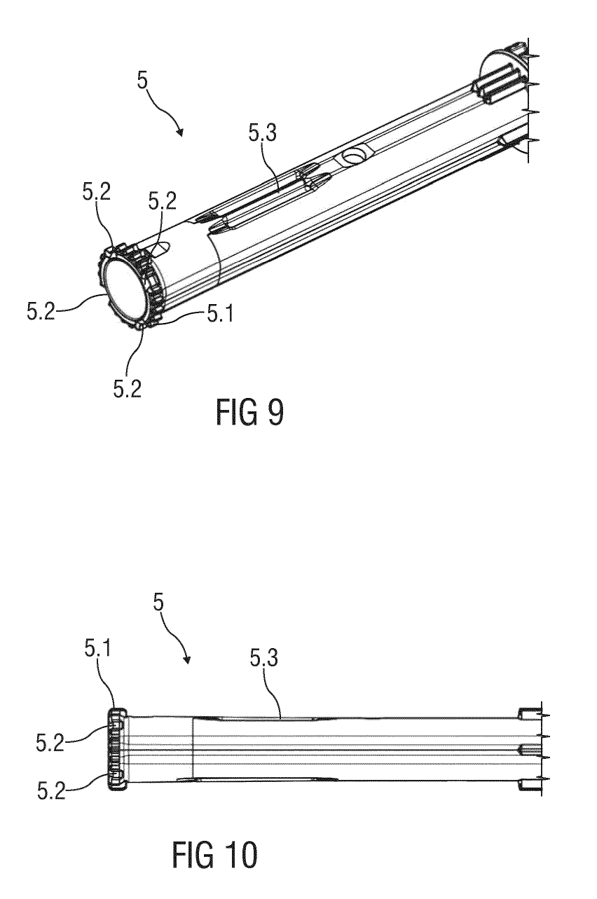

The FIGS. 9 and 10 show an exemplary embodiment of the drive sleeve 5 in different views. FIG. 9 shows a perspective view of the drive sleeve 5 and FIG. 10 shows a side view of the drive sleeve 5.

The drive sleeve 5 is rotationally locked to the piston rod 4 via a spline (not shown) on the inside of the drive sleeve 5. In order to reduce material thickness, a recess is formed on the outside of the drive sleeve 5 following the profile of the spline. When the round torsional drive spring 9 is charged, its diameter reduces and its coils may contact the distal or proximal ends of the recess, increasing friction between the round torsional drive spring 9 and the drive sleeve 5. In order to reduce this friction, the drive sleeve 5 comprises a rib 5.3 that extends axially on an outer circumference of the drive sleeve 5. The rib 5.3 provides a smooth and substantially circular contact surface for the round torsional drive spring 9.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,
H-(Lys)5-des Pro36, des Pro37 Exendin-4(1-39)-NH2,
des Pro36 Exendin-4(1-39),
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28]
Exendin-4(1-39),
des Pro36 [Met(O)14 Trp(02)25, IsoAsp28]
Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28]
Exendin-4(1-39),
des Pro36 [Met(O)14 Trp(O2)25, IsoAsp28]
Exendin-4(1-39),

wherein the group -Lys6-NH2 may be bound to the C-terminus of the Exendin-4 derivative;
or an Exendin-4 derivative of the sequence

des Pro36 Exendin-4(1-39)-Lys6-NH2 (AVE0010),
H-(Lys)6-des Pro36 [Asp28]
Exendin-4(1-39)-Lys6-NH2,
des Asp28 Pro36, Pro37, Pro38Exendin-4(1-39)-NH2,
H-(Lys)6-des Pro36, Pro38 [Asp28]
Exendin-4(1-39)-NH2,
H-Asn-(Glu)5des Pro36, Pro37, Pro38 [Asp28]
Exendin-4(1-39)-NH2,
des Pro36, Pro37, Pro38 [Asp28]
Exendin-4(1-39)-(Lys)6-NH2,
H-(Lys)6-des Pro36, Pro37, Pro38 [Asp28]
Exendin-4(1-39)-(Lys)6-NH2,
H-Asn-(Glu)5-des Pro36, Pro37, Pro38 [Asp28]
Exendin-4(1-39)-(Lys)6-NH2,
H-(Lys)6-des Pro36 [Trp(O2)25, Asp28]
Exendin-4(1-39)-Lys6-NH2,
H-des Asp28 Pro36, Pro37, Pro38 [Trp(O2)25]
Exendin-4(1-39)-NH2,
H-(Lys)6-des Pro36, Pro37, Pro38 [Trp(O2)25,
Asp28] Exendin-4(1-39)-NH2,
H-Asn-(Glu)5-des Pro36, Pro37, Pro38
[Trp(O2)25, Asp28] Exendin-4(1-39)-NH2,
des Pro36, Pro37, Pro38 [Trp(O2)25, Asp28]
Exendin-4(1-39)-(Lys)6-NH2,
H-(Lys)6-des Pro36, Pro37, Pro38 [Trp(O2)25,
Asp28] Exendin-4(1-39)-(Lys)6-NH2,
H-Asn-(Glu)5-des Pro36, Pro37, Pro38 [Trp(O2)25,
Asp28] Exendin-4(1-39)-(Lys)6-NH2,
H-(Lys)6-des Pro36 [Met(O)14, Asp28]
Exendin-4(1-39)-Lys6-NH2,
des Met(O)14 Asp28 Pro36, Pro37, Pro38
Exendin-4(1-39)-NH2,
H-(Lys)6-desPro36, Pro37, Pro38 [Met(O)14,
Asp28] Exendin-4(1-39)-NH2,
H-Asn-(Glu)5-des Pro36, Pro37, Pro38 [Met(O)14,
Asp28] Exendin-4(1-39)-NH2,
des Pro36, Pro37, Pro38 [Met(O)14, Asp28]
Exendin-4(1-39)-(Lys)6-NH2,
H-(Lys)6-des Pro36, Pro37, Pro38 [Met(O)14,
Asp28] Exendin-4(1-39)-(Lys)6-NH2,
H-Asn-(Glu)5 des Pro36, Pro37, Pro38 [Met(O)14,
Asp28] Exendin-4(1-39)-(Lys)6-NH2,
H-Lys6-des Pro36 [Met(O)14, Trp(O2)25, Asp28]
Exendin-4(1-39)-Lys6-NH2,
H-des Asp28 Pro36, Pro37, Pro38 [Met(O)14,
Trp(O2)25] Exendin-4(1-39)-NH2,
H-(Lys)6-des Pro36, Pro37, Pro38 [Met(O)14,
Asp28] Exendin-4(1-39)-NH2,
H-Asn-(Glu)5-des Pro36, Pro37, Pro38 [Met(O)14,
Trp(O2)25, Asp28] Exendin-4(1-39)-NH2,
des Pro36, Pro37, Pro38 [Met(O)14, Trp(O2)25,
Asp28] Exendin-4(1-39)-(Lys)6-NH2,
H-(Lys)6-des Pro36, Pro37, Pro38 [Met(O)14,
Trp(O2)25, Asp28] Exendin-4(S1-39)-(Lys)6-NH2,
H-Asn-(Glu)5-des Pro36, Pro37, Pro38 [Met(O)14,
Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two p sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains p and E have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

Those of skill in the art will understand that modifications (additions and/or removals) of various components of the apparatuses, methods and/or systems and embodiments described herein may be made without departing from the full scope and spirit of the present disclosure, which encompass such modifications and any and all equivalents thereof.

### LIST OF REFERENCES

1 drug delivery device
2 housing
2.1 first restraining element
2.1.1 first fixing recess
2.1.2 first axial protrusions
3 cartridge holder
4 piston rod
5 drive sleeve
5.1 splined teeth
5.2 assembly recess
5.3 rib
6 number sleeve
6.1 second restraining element
6.1.1 second fixing recess
6.1.2 second axial protrusions
7 button
8 dose selector
9 round torsional drive spring
9.1 distal spring end
9.2 proximal spring end
10 cartridge
11 gauge element
12 clutch element
13 clutch spring
14 bearing
15 stopper
A longitudinal axis

1. A drug delivery device for dispensing a dose of a liquid medicament, the drug delivery device comprising:
a rotationally driveable expelling mechanism configured to expel the dose of the liquid medicament from a medicament container;
a round torsional drive spring configured to apply a driving torque to the rotationally driveable expelling mechanism when energized by an external strain, wherein the round torsional drive spring encloses an interior space of varying shape a or extension according to the level of external strain applied to the round torsional drive spring; and
at least one restraining element defining a number of rest surfaces to internally support the round torsional drive spring increasingly with the level of external strain applied thereto, wherein the shape of the rest surfaces is such as to essentially maintain the center of the interior space throughout the entire variation in external strain applicable to the round torsional drive spring.

2. The drug delivery device according to claim 1, wherein the rotationally driveable expelling mechanism comprises a threaded piston rod and a drive sleeve, wherein the drive sleeve is configured, when rotationally driven, to cause the piston rod to rotate relatively to threaded member, wherein the threaded member is provided in fixed axial position relative to the medicament container and is in engagement with the thread of the piston rod.

3. The drug delivery device according to claim 1, wherein the round torsional drive spring extends in an axial direction and is eccentrically fixed with respect to a longitudinal axis of the drug delivery device, wherein one of the spring ends is arranged on the at least one restraining element and is fixed at a mounting point, and the at least one restraining element tapers in the axial direction from the mounting point.

4. The drug delivery device according to claim 1, wherein, during tensioning of the round torsional drive spring, the one or more restraining elements are configured to partially space apart the round torsional drive spring from the drive sleeve.

5. The drug delivery device according to claim 1, wherein the at least one restraining element comprises at least one axially extending projection.

6. The drug delivery device according to claim 5, wherein the projection protrudes in parallel to a torque axis of the round torsional drive spring.

7. The drug delivery device according to claim 5, wherein an outer diameter of the projection decreases from the mounting point towards a tip of the projections.

8. The drug delivery device according to claim 1, wherein a diameter of the interiors space enclosed by the round torsional drive spring decreases during dose setting, whereby the round torsional drive spring is being stressed.

9. The drug delivery device according to claim 8, wherein the round torsional drive spring is helically coiled and one or more spring coils are supported by one or more tapered steps of the restraining element.

10. The drug delivery device according to claim 1, wherein the at least one restraining element comprises two to six axial projections distributed about a circumference of the at least one restraining element.

11. The drug delivery device according to claim 1, wherein a first restraining element is coupled to or configured as a part of a housing.

12. The drug delivery device according to claim 11, the first restraining element fixes a distal spring end of the round torsional drive spring.

13. The drug delivery device according to claim 1, wherein a second restraining element is coupled to or configured as a part of a number sleeve.

14. The drug delivery device according to claim 13, wherin the second restraining element fixes a proximal spring end.

15. The drug delivery device according to claim 14, wherein the round torsional drive spring is stressed due to a rotational movement of a number sleeve with respect to a housing.

16. The drug delivery device according to claim 15, wherein at least one of the spring coils of the round torsional drive spring abuts against the axial projections when the round torsional drive spring is strained.

17. The drug delivery device (1) according to claim 1, wherein the round torsional drive spring is released during dispensing the dose of medicament, wherein the drive sleeve rotates together with the number sleeve with respect to the housing due to the release of the round torsional drive spring.
